(19) United States
(12) Patent Application Publication
(10) Pub. No.: US 2022/0225926 A1
(43) Pub. Date: Jul. 21, 2022

(54) EPILEPSY MONITORING DEVICE AND EPILEPSY MONITORING SYSTEM

(71) Applicants: EWHA UNIVERSITY - INDUSTRY COLLABORATION FOUNDATION, Seoul (KR); KOREA ADVANCED INSTITUTE OF SCIENCE AND TECHNOLOGY, Daejeon (KR)

(72) Inventors: Hyang Woon LEE, Seoul (KR); Je Won KANG, Seoul (KR); Ji Hoon KIM, Seoul (KR); Min Kyu JE, Daejeon (KR); Seung Kyun KANG, Daejeon (KR)

(21) Appl. No.: 17/595,638

(22) PCT Filed: May 6, 2020
(86) PCT No.: PCT/KR2020/005957

§ 371 Date: Mar. 29, 2022

(30) Foreign Application Priority Data
May 20, 2019 (KR) 10-2019-0059123

(51) Int. Cl. A61B 5/369; A61B 5/00

(52) U.S. Cl. CPC A61B 5/369; A61B 5/7275; A61B 5/746; A61B 2562/227; A61B 5/4836; A61B 5/7264; A61B 5/0006; A61B 5/6814

(57) ABSTRACT

This application relates to an epilepsy monitoring device. In one aspect, the device includes a first unit having a first body portion arranged on a user's head, a sensor unit that is connected to the first body portion and includes a first sensor for measuring a cranial nerve signal, and a stimulation unit that is connected to the first body portion and applies cranial nerve treatment stimulation to the brain according to a provided brain stimulation signal. The device may also include a second unit electrically connected to the first unit and having a second body portion arranged on a body other than the user's head, a battery unit that is arranged in the second body portion and supplies power to the first unit, and a communication unit for wireless communication with an external device.

### TECHNICAL FIELD

Embodiments of the present invention relate to an epilepsy monitoring device and an epilepsy monitoring system.

### BACKGROUND ART

An epileptic seizure causes changes in behavior or consciousness, such as losing consciousness or shaking limbs violently for as short as a few seconds or minutes during the seizure. Epilepsy is usually a disease of the brain that normally functions abnormally. Epilepsy is accompanied by unfamiliar feelings, emotions, behaviors, or sometimes seizures such as convulsions, muscle spasms, and loss of consciousness. Some people with epilepsy have seizures very infrequently, while others have more than 100 seizures per day. The number of seizures varies from person to person, and the risk of seizures varies. The risk of seizures is higher if a person with an epileptic seizure has a medical condition such as hypoxia (chronic obstructive pulmonary disease or severe asthma), meningitis (meningitis), encephalitis, or a brain tumor.

It is known that there are more than 2 million patients with epilepsy in the United States, and it is reported that 80% of these epilepsy patients can control their seizures by medication and surgery. However, the remaining 25 to 30% of patients continue to experience seizures. In the UK, there are 600,000 people with epilepsy. Of these, about 500 patients are injured by sudden seizures and die from such injuries. In the case of Korea, there are no accurate statistics, but it is estimated that there are about 300,000 to 400,000 epilepsy patients.

In addition, electroencephalography (EEG) is measured using electrodes to measure the current flow in the living body caused by the synchronized activity of nerve cells occurring on the surface of the brain cortex, and can be measured by attaching electroencephalography electrodes to the skin of the scalp or inserting an electroencephalography electrode into the cranial cavity by surgery. However, because the conventional monitoring device is formed as an integrated battery, additional brain surgery is required to replace the battery after insertion into the body, and there is a problem in that it is impossible to take a magnetic resonance image (MRI) while the monitoring device is inserted into the body. In addition, conventionally, epilepsy data analysis using electroencephalography has been used for epilepsy diagnosis, convulsion detection and prediction, but it takes a long time because electroencephalography data for all or a wide range of frequencies are precisely analyzed from all electroencephalography electrodes before and after convulsions occur. Therefore, it is difficult to detect convulsions in the early onset of convulsions.

### DESCRIPTION OF EMBODIMENTS

### Technical Problem

In order to solve the above problems, embodiments of the present invention provide an epilepsy monitoring device separated into a first unit having only essential components for electroencephalography measurement and brain stimulation, and a second unit including the remaining components, and an epilepsy monitoring system including the same.

### Solution to Problem

One embodiment of the present invention provides an epilepsy monitoring device including a first unit having a first body portion arranged on a user's head, a sensor unit that is connected to the first body portion and includes a first sensor for measuring a cranial nerve signal, and a stimulation unit that is connected to the first body portion and applies cranial nerve treatment stimulation to the brain according to a provided brain stimulation signal, and a second unit electrically connected to the first unit and having a second body portion arranged on a body other than the user's head, a battery unit that is arranged in the second body portion and supplies power to the first unit, and a communication unit for wireless communication with an external device.

### Advantageous Effects of Disclosure

An epilepsy monitoring device and an epilepsy measurement system according to embodiments of the present invention have the advantage that a battery can be replaced without additional surgical treatment through a dual structure of a first unit and a second unit, and medical imaging such as magnetic resonance imaging is possible even when the epilepsy monitoring device is inserted into the user's brain. In addition, an epilepsy monitoring device and an epilepsy monitoring system according to embodiments of the present invention may achieve a high level of detection and prediction accuracy by generating a user-customized convulsion detection and prediction algorithm through artificial intelligence, and periodically updating the algorithm through provided data.

### BRIEF DESCRIPTION OF DRAWINGS

FIG. 1 is a view illustrating an example of a network environment according to an embodiment of the present invention.

FIG. 2 is a view for explaining an epilepsy monitoring device according to an embodiment of the present invention.

FIG. 3 is a block diagram of the epilepsy monitoring device of FIG. 2.

FIG. 4 is a block diagram of the epilepsy monitoring device of FIG. 2 according to another embodiment.

FIG. 5 is a view for explaining an epilepsy monitoring system including the epilepsy monitoring device of FIG. 2.

FIG. 6 is a view for explaining a process of generating a user-customized convulsion detection and prediction algorithm in a learning unit.

FIG. 7 is a view for explaining a process of determining whether a convulsion occurs using a determination unit.

### BEST MODE

One embodiment of the present invention provides an epilepsy monitoring device including a first unit having a first body portion arranged on a user's head, a sensor unit that is connected to the first body portion and includes a first sensor for measuring a cranial nerve signal, and a stimulation unit that is connected to the first body portion and applies cranial nerve treatment stimulation to the brain according to a provided brain stimulation signal, and a second unit electrically connected to the first unit and having a second body portion arranged on a body other than the user's head, a battery unit that is arranged in the second body portion and supplies power to the first unit, and a communication unit for wireless communication with an external device.

In an embodiment of the present invention, the second unit may further include a determination unit that determines whether the user has convulsions according to the cranial nerve signal using a previously stored user-customized convulsion detection and prediction algorithm, and generates an alarm signal when it is determined that the user has convulsions.

In an embodiment of the present invention, the determination unit may generate the brain stimulation signal to apply the cranial nerve treatment stimulation corresponding to the brain stimulation signal to the user's brain, and provide the brain stimulation signal to the stimulation unit.

In an embodiment of the present invention, the communication unit may include a first communication means for communicating with an external device by a remote active communication method, and a second communication means for communicating with an external device by a proximity passive communication method.

In an embodiment of the present invention, the first communication means may transmit and receive the alarm signal to an external device by a remote active communication method, and the second communication means may transmit and receive the cranial nerve signal or the brain stimulation signal to an external device by a proximity passive communication method.

In an embodiment of the present invention, the sensor unit may further include a second sensor for detecting a biological signal different from the cranial nerve signal.

In an embodiment of the present invention, the second sensor may detect a user's motion signal.

One embodiment of the present invention provides an epilepsy monitoring system including a first unit having a first body portion arranged on a user's head, a sensor unit that is connected to the first body portion and includes a first sensor for measuring a cranial nerve signal, and a stimulation unit that is connected to the first body portion and applies cranial nerve treatment stimulation to the brain according to a provided brain stimulation signal, a second unit electrically connected to the first unit and having a second body portion arranged on a body other than the user's head, a battery unit that is arranged in the second body portion and supplies power to the first unit, and a communication unit for wireless communication with an external device, a learning unit for machine learning a user-customized convulsion detection and prediction algorithm using a normal cranial nerve signal and a convulsive cranial nerve signal from among pre-obtained cranial nerve signals of the user, and a determination unit that determines whether the user has convulsions using the user-customized convulsion detection and prediction algorithm and the cranial nerve signals measured in real time by the sensor unit.

In an embodiment of the present invention, the determination unit is arranged in the second unit, and may generate an alarm signal when it is determined that the user has convulsions.

In an embodiment of the present invention, the determination unit may generate the brain stimulation signal to apply the cranial nerve treatment stimulation corresponding to the brain stimulation signal to the user's brain, and provide the brain stimulation signal to the stimulation unit.

In an embodiment of the present invention, the communication unit may include a first communication means for communicating with an external device by a remote active communication method, and a second communication means for communicating with an external device by a proximity passive communication method.

In an embodiment of the present invention, the first communication means may transmit and receive the alarm signal to an external device by a remote active communication method, and the second communication means may transmit and receive the cranial nerve signal or the brain stimulation signal to an external device by a proximity passive communication method.

In an embodiment of the present invention, the sensor unit may further include a second sensor for detecting a biological signal different from the cranial nerve signal.

In an embodiment of the present invention, the second sensor may detect a user's motion signal.

Additional aspects will be set forth in part in the description which follows and, in part, will be apparent from the description, or may be learned by practice of the presented embodiments.

### MODE OF DISCLOSURE

Since the present invention may have diverse modified embodiments, preferred embodiments are illustrated in the drawings and are described in the detailed description. An effect and a characteristic of the present invention, and a method of accomplishing these will be apparent when referring to embodiments described with reference to the drawings. The present invention may, however, be embodied in many different forms and should not be construed as limited to the embodiments set forth herein.

Hereinafter, embodiments of the present invention will be described in detail with reference to the accompanying drawings. The same reference numerals are used to denote the same elements, and repeated descriptions thereof will be omitted.

It will be understood that although the terms “first,” “second,” etc. may be used herein to describe various components, these components should not be limited by these terms.

An expression used in the singular encompasses the expression of the plural, unless it has a clearly different meaning in the context.

It will be further understood that the terms “comprises” and/or “comprising” used herein specify the presence of stated features or elements, but do not preclude the presence or addition of one or more other features or elements.

It will be understood that when a layer, region, or element is referred to as being “formed on” another layer, area, or element, it can be directly or indirectly formed on the other layer, region, or element. That is, for example, intervening layers, regions, or elements may be present.

Sizes of elements in the drawings may be exaggerated for convenience of explanation. In other words, since sizes and thicknesses of components in the drawings are arbitrarily illustrated for convenience of description, the following embodiments are not limited thereto.

When a certain embodiment may be implemented differently, a specific process order may be performed differently from the described order. For example, two consecutively described processes may be performed substantially at the same time or performed in an order opposite to the described order.

It will be understood that when a layer, region, or component is connected to another portion, the layer, region, or component may be directly connected to the portion or an intervening layer, region, or component may exist. For example, when a layer, region, or component is electrically connected to another portion, the layer, region, or component may be directly electrically connected to the portion or may be indirectly connected to the portion through another layer, region, or component.

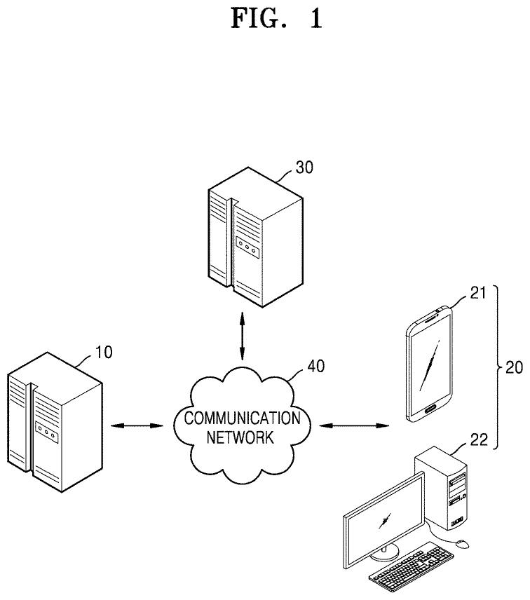

FIG. 1 is a view illustrating an example of a network environment according to an embodiment of the present invention.

The network environment of FIG. 1 shows an example including a user terminal 20, servers 10 and 30, and a network 40. FIG. 1 is an example for explaining the invention, and the number of user terminals or the number of servers is not limited as in FIG. 1.

The user terminal 20 may be a fixed terminal 22 implemented as a computer device or a mobile terminal 21. The user terminal 20 may be a terminal for transmitting data received from an epilepsy measuring device 100 to be described later below to the servers 10 and 30. Examples of the user terminal 20 include a smartphone, a mobile phone, a navigation, a computer, a laptop, a digital broadcasting terminal, personal digital assistants (PDA), a portable multimedia player (PMP), a tablet PC, and the like. For example, a first user terminal 21 may communicate with another user terminal 22 and/or the servers 10 and 30 through the network 40 using a wireless or wired communication method.

The communication method is not limited thereto, and not only a communication method (e.g., a mobile communication network, wired Internet, wireless Internet, or a broadcasting network) using a communication network that the network 40 may include, but also short-range wireless communication between devices may be included. For example, the network 40 may include any one or more of networks such as a personal area network (PAN), local area network (LAN), campus area network (CAN), metropolitan area network (MAN), wide area network (WAN), a broadband network (BBN), the Internet, and the like. In addition, the network 40 may include any one or more of a network topology including, but not limited to, a bus network, a star network, a ring network, a mesh network, a star-bus network, a tree or a hierarchical network, and the like.

The servers 10 and 30 may be implemented as a computer device or a plurality of computer devices that communicate with the user terminal 20 through the network 40 to provide commands, code, files, content, services, and the like.

For example, the servers 10 and 30 may provide a file for installing an application to the first user terminal 21 connected through the network 40. In this case, the first user terminal 21 may install an application using files provided from the servers 10 and 30. In addition, the first user terminal 21 may access the servers 10 and 30 under the control of an operating system (OS) and at least one program (e.g., a browser or an installed application) included in the first user terminal 21 to receive services or content provided by the servers 10 and 30. As another example, the servers 10 and 30 may establish a communication session for data transmission/reception, and route data transmission/reception between user terminals 20 through the established communication session.

According to an embodiment of the present invention, the servers 10 and 30 may receive a user's cranial nerve signal through the user terminal 20 and generate a user-customized convulsive ELECTROENCEPHALOGRAPHY detection and prediction algorithm based on the cranial nerve signal and deep learning. In addition, the servers 10 and 30 may generate a user-customized treatment stimulation signal generating algorithm by using the user's cranial nerve signals and deep learning provided from the user terminal 20 before and after application of treatment stimulation to the brain. The user terminal 20 may periodically transmit a user's cranial nerve signal received from the epilepsy measuring device 100 to the servers 10 and 30, and the servers 10 and 30 may update the user-customized convulsive ELECTROENCEPHALOGRAPHY detection and prediction algorithm and the user-customized treatment stimulation signal generating algorithm according to the periodically transmitted cranial nerve signal.

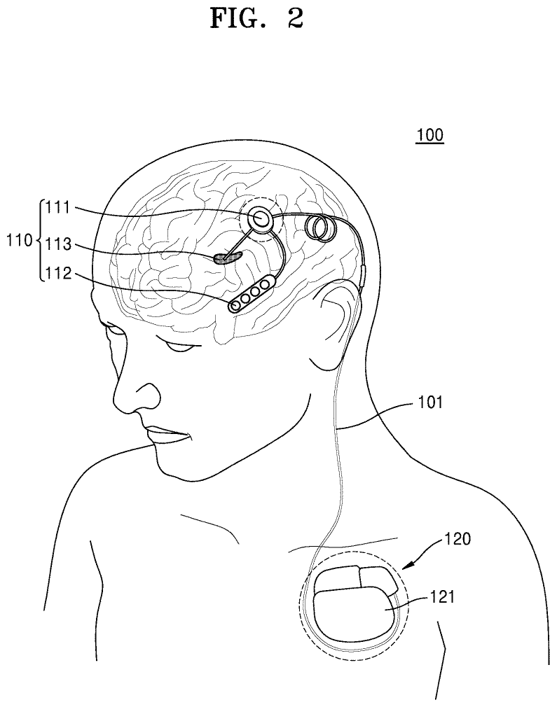

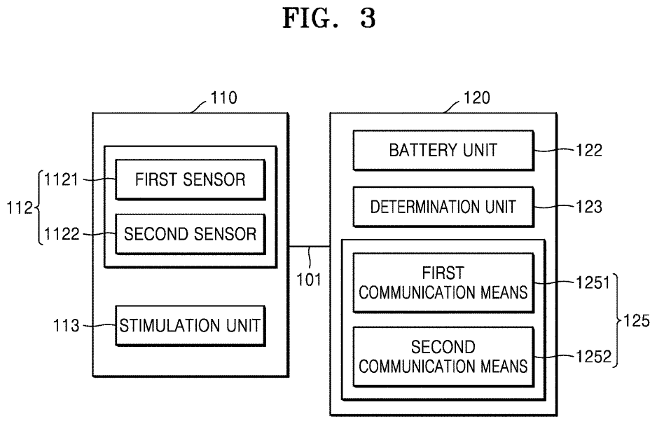

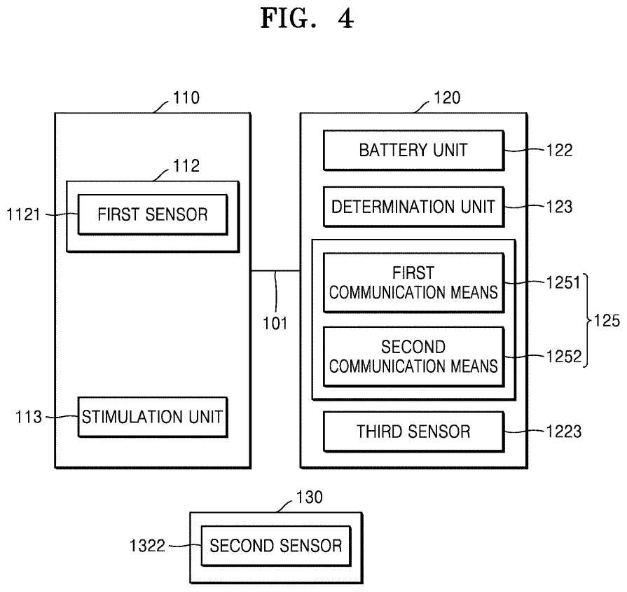

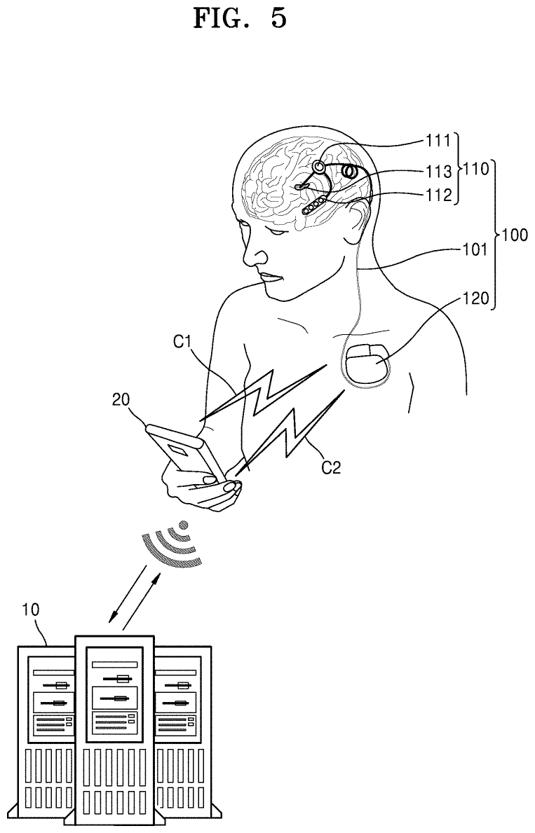

FIG. 2 is a view for explaining the epilepsy monitoring device 100 according to an embodiment of the present invention, and FIG. 3 is a block diagram of the epilepsy monitoring device 100 of FIG. 2. FIG. 4 is a block diagram of the epilepsy monitoring device 100 of FIG. 2 according to another embodiment, and FIG. 5 is a view for explaining an epilepsy monitoring system including the epilepsy monitoring device 100 of FIG. 2.

Referring to FIGS. 2 and 3, the epilepsy measuring device 100 according to an embodiment of the present invention may include a first unit 110 arranged on a user's head and a second unit 120 arranged on a body other than the user's head.

The epilepsy measuring device 100 according to an embodiment of the present invention is characterized in that it has a dual structure in which only an essential component consisting of a sensor unit 112 for measuring a cranial nerve signal and a stimulation unit 113 for applying cranial nerve treatment stimulation to the brain is placed on a user's head, and components such as the remaining battery unit 122 and a processor for data processing are placed on another body apart from the user's head.

The epilepsy measuring device 100 should be arranged adjacent to the user's brain to receive a fine cranial nerve signal for accurate epilepsy detection and prediction. The conventional epilepsy measuring device includes a power supply means such as a battery to supply power to a sensor for measuring a cranial nerve signal or a stimulation means for applying a therapeutic stimulus to the brain. However, because the power supply means such as a battery contains a magnetic material, medical images such as magnetic resonance imaging (MRI) cannot be measured with an epilepsy measuring device inserted into the body, and surgery is required to replace the battery.

Because the epilepsy measuring device 100 according to an embodiment of the present invention separates the first unit 110 arranged on the user's head and the second unit 120 arranged on a body other than the user's head and forms a dual structure that minimizes a configuration to be arranged on the first unit 110, it is possible to measure a medical image such as a magnetic resonance image while the epilepsy measuring device 100 is inserted into the user's head, and a battery may be replaced without additional surgery.

Hereinafter, each component of the epilepsy measuring device 100 will be described in more detail.

The first unit 110 includes a first body portion 111, the sensor unit 112, and the stimulation unit 113.

The first body portion 111 is arranged on the user's head, and may be arranged outside the skull. The first body portion 111 may be formed of a nonferrous and nonferromagnetic material so that magnetic resonance imaging (MRI) may be performed even when implanted in the body. For example, the first body portion 111 may be made of a material such as reinforced plastic, austenite cast iron, or silicon.

In addition, although not shown, an auxiliary circuit (not shown) for controlling the sensor unit 112 and the stimulation unit 113 may be inserted into the first body portion 111. At this time, the auxiliary circuit (not shown) controls the sensor unit 112 and the stimulation unit 113 to receive or transmit data, but the actual control is processed by a processor arranged in the second unit 120, so the auxiliary circuit may include a minimum circuit for controlling the sensor unit 112 and the stimulation unit 113. The auxiliary circuit (not shown) may include a low-power and high-performance integrated circuit device, and may be made of a nonferrous and nonferromagnetic material so that interference to magnetic resonance imaging (MRI) does not occur when implanted in the body.

The sensor unit 112 may be a means for measuring a biological signal including a user's cranial nerve signal. The sensor unit 112 may include a first sensor 1121 connected to the first body portion 111 and arranged adjacent to the user's brain to measure a cranial nerve signal. The first sensor 1121 may be inserted into the cranial cavity, and the insertion position may vary depending on the user's lesion characteristics. The first sensor 1121 may be electrically connected to a control circuit (not shown) arranged on the first body portion 111 to transmit a cranial nerve signal sensed in real time at the insertion position to the control circuit (not shown). As shown, the first sensor 1121 may be an electrode that is inserted into the cranial cavity and arranged on the surface of the brain.

Meanwhile, the sensor unit 112 may further include a second sensor 1122 for measuring a biological signal different from the cranial nerve signal. The second sensor 1122 may be a motion sensor that generates a motion signal by detecting a biological signal different from that of the first sensor 1121, for example, whether a user is in motion. In other words, the sensor unit 112 not only senses a cranial nerve signal to detect whether a user has convulsions, but also simultaneously senses other biological signals in addition to the cranial nerve signal to more accurately determine whether the user has convulsions. In more detail, a determination unit 123 to be described later below determines whether a user has convulsions by using user's real-time cranial nerve signals and a user-customized convulsion detection and prediction algorithm. At this time, the determination unit 123 may accurately determine whether the user is actually convulsed according to whether the user has collapsed through a motion signal sensed together.

As another embodiment, as shown in FIG. 4, a second sensor 1322 may not be arranged in the first unit 110, but may be arranged in another third unit 130 physically separated from the first unit 110. In more detail, the second sensor 1322 may be a motion sensor as described above, and may be arranged on any part capable of detecting the user's collapse even if the head is not moved. Accordingly, the second sensor 1322 may be physically separated from the first unit 110 located on the user's head, and may be included in the third unit 130 located on the user's other body. For example, the third unit 130 may be a wristband worn on the user's wrist.

Referring again to FIGS. 2 and 3, the stimulation unit 113 is connected to the first body portion 111 and may apply cranial nerve treatment stimulation to the brain according to a brain stimulation signal provided from the external servers 10 and 30 or the determination unit 123 of the second unit 120. The stimulation unit 113 may be an electrode inserted up to a pre-set lesion to electrically or magnetically stimulate a spasmodic lesion or a specific brain region. For the treatment of chronic brain diseases, deep brain stimulation (DBS), which electrically stimulates the thalamus or hypothalamic nucleus, electrical cortical stimulation (ECS), which stops convulsive seizures and suppresses the occurrence of spontaneous convulsive seizures by electrical or magnetic stimulation of convulsive lesions or specific brain regions, or repetitive transcranial magnetic stimulation (rTMS) may be used. In this case, a user's convulsive lesion or a specific brain region may be located at a deep position in the brain, and the stimulation unit 113 may be formed of a depth electrode capable of delivering therapeutic stimulation up to the above position.

The stimulation unit 113 is electrically connected to the control circuit (not shown) arranged on the first body portion 111 like the sensor unit 112, and may apply cranial nerve treatment stimulation to the specific brain region in response to the brain stimulation signal provided from the external server 10 or 30 or the determination unit 123 of the second unit 120.

The second unit 120 includes a second body portion 121, the battery unit 122, and a communication unit 125. The first unit 110 may be electrically connected to the second unit 120 through an electric wire 101 as shown.

The second body portion 121 may be arranged on a body other than the user's head. For example, the second body portion 121 may be arranged on the chest of the user as shown.

The battery unit 122 is arranged on the second body portion 121 and may supply power not only to a driving device included in the second unit 120, but also to the first unit 110. The battery unit 122 may use a replaceable battery, but is not limited thereto, and may use a rechargeable battery.

The communication unit 125 is arranged on the second body portion 121 and may perform a function for wireless communication with an external device, for example, the user terminal 20. At this time, the communication unit 125 includes a first communication means 1251 by a remote active communication method and a second communication means 1252 by a proximity passive communication method, and may communicate with an external device using both the wireless communication methods.

When it is determined as convulsions according to a measured cranial nerve signal, the epilepsy measuring device 100 may generate an alarm signal and inform a user, or may transmit the measured cranial nerve signal to the external servers 10 and 30 to periodically update a user-customized convulsion detection and prediction algorithm.

In this case, when the communication unit 125 uses a remote active communication method such as Bluetooth, ZigBee, or a Medical Implant Communication Service (MISC), in the case of single-shot data such as an alarm signal, transmission is possible, but in order to transmit an accumulated cranial nerve signal to the outside, power consumption becomes excessively large, making it difficult to transmit the cranial nerve signal to the outside. Alternatively, when the communication unit 125 uses a proximity passive communication method such as Near Field Communication (NFC), it is only necessary to bring the user terminal 20 into proximity, so that the cranial nerve signal may be transmitted without battery consumption. However, because only proximity communication is possible, there is a problem in that it is difficult to transmit a real-time alarm signal when convulsions are detected.

Therefore, the epilepsy measuring device 100 according to an embodiment of the present invention includes the first communication means 1251 by the remote active communication method and the second communication means 1252 by the proximity passive communication method to solve the above problems, and may communicate with an external device using both the wireless communication methods. In addition, the communication unit 125 may transmit/receive a brain stimulation signal from the epilepsy measuring device 100 to the user terminal 20.

On the other hand, as another embodiment, referring to FIG. 4, the second unit 120 may further include a third sensor 1223 for detecting a biological signal different from a user's cranial nerve signal. In this case, the third sensor 1223 may be an electrocardiogram sensor for measuring an electrocardiogram (ECG) signal. The epilepsy measuring device 100 according to another embodiment measures a motion signal or an ECG signal together with a cranial nerve signal, so that the determination unit 123 to be described later below may more accurately detect and predict whether convulsions occur.

Hereinafter, an epilepsy measuring system will be described in more detail.

Referring to FIGS. 2 to 5, the epilepsy measuring system may include the epilepsy measuring device 100 including the above-described first unit 110 and the second unit 120, a learning unit, and the determination unit 123.

Here, the epilepsy measuring system may include at least one processor. Accordingly, the epilepsy measuring system may be driven in a form included in a hardware device such as a microprocessor or a general-purpose computer system. Here, the processor may refer to, for example, a data processing device embedded in hardware having a physically structured circuit to perform a function expressed as code or an instruction included in a program. As an example of the data processing device embedded in the hardware as described above, processing devices such as a microprocessor, a central processing unit (CPU), a processor core, a multiprocessor, an application-specific integrated circuit (ASIC), and a field programmable gate array (FPGA) may be included, but the scope of the present invention is not limited thereto.

For example, a request generated by a processor of the epilepsy measuring device 100 according to program code stored in a recording device such as a memory may be transmitted to the user terminal 20 under the control of a communication unit. A processor of the user terminal 20 may also transmit the request generated according to the program code stored in the recording device such as a memory to the servers 10 and 30 through the network 40 under the control of a communication module. Conversely, a control signal, a command, content, or file provided under the control of a processor of the servers 10 and 30 is received by the user terminal 20 through the communication module of the user terminal 20 via the network 40, and may be transmitted to the epilepsy measuring device 100 through the communication unit. For example, a user-customized convulsion detection and prediction algorithm of the servers 10 and 30 received through the communication module may be transmitted to the processor or memory of the epilepsy measuring device 100.

The processor may be configured to process commands of a computer program by performing basic arithmetic, logic, and input/output operations. The commands may be provided to the processor by a memory or a receiver. For example, the processor may be configured to execute received commands according to program code stored in a recording device, such as a memory. The processor of the epilepsy measuring device 100 may include the determination unit 123.

As an embodiment, the learning unit may be mounted on the external server 10, and the determination unit 123 may be provided in the epilepsy measuring device 100, more specifically, in the second unit 120 of the epilepsy measuring device 100. That is, a process of generating a user-customized convulsion detection and prediction algorithm may be performed in the external server 10, and a process of determining whether convulsions occur using the user-customized convulsion detection and prediction algorithm and a cranial nerve signal sensed in real time may be performed in the second unit 120 of the epilepsy measuring device 100. However, the present invention is not limited thereto, and the learning unit and the determination unit 123 may be provided in one device to perform both learning and determining functions.

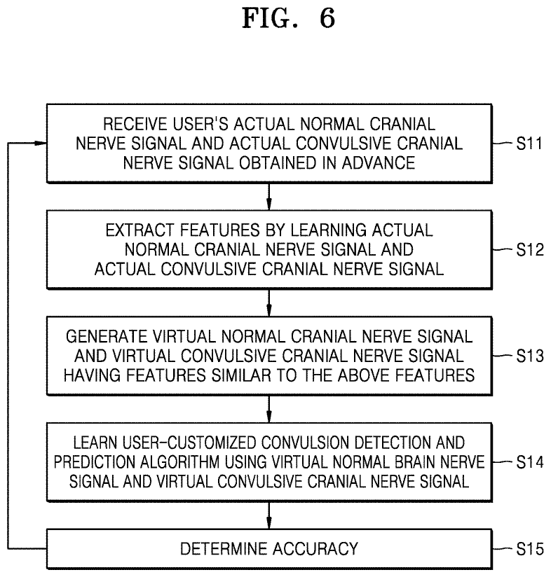

Hereinafter, a process of generating a user-customized convulsion detection and prediction algorithm in a learning unit will be described with reference to FIG. 6.

FIG. 6 is a view for explaining a process of generating a user-customized convulsion detection and prediction algorithm in a learning unit.

Referring to FIG. 6, first, in operation S11, the learning unit receives a user's actual normal cranial nerve signal and an actual brain spasm signal obtained in advance. At this time, the user's actual normal cranial nerve signal and the actual brain spasm signal may be cranial nerve signals measured from the epilepsy measuring device 100, or may be signals that are identified and classified through a result of whether the user actually has convulsions.

Next, in operation S12, the learning unit extracts features by learning an actual normal cranial nerve signal and an actual convulsive cranial nerve signal. As an embodiment, the learning unit may calculate power spectrum density (PSD) of cranial nerve signals received in time series, and may analyze the PSD to detect a specific frequency band including a frequency having the greatest separation width between the actual normal cranial nerve signal and the actual convulsive cranial nerve signal. For example, the specific frequency band may be a frequency band having a certain range based on the frequency having the greatest separation width. Here, the specific frequency band may be detected using a Welch method. However, the present invention is not limited thereto, and the learning unit may extract not only the specific frequency band but also other features from the actual normal cranial nerve signal and the actual cranial nerve signal.

Next, in operation S13, the learning unit generates a virtual normal cranial nerve signal and a virtual convulsive cranial nerve signal having features similar to the extracted features. The user's actual normal cranial nerve signal and the actual convulsive cranial nerve signal may be obtained after a user wears the epilepsy measuring device 100. In the case of the user's actual normal cranial nerve signal and the actual convulsive cranial nerve signal, the number of data may be insufficient in performing machine learning because the frequency of actual convulsions is not so high. The learning unit, using the features extracted from the actual normal cranial nerve signal and the actual cranial nerve signal as described above, may generate a virtual normal cranial nerve signal and a virtual convulsive cranial nerve signal having features similar to the actual normal cranial nerve signal and the actual cranial nerve signal characteristics and use the virtual normal cranial nerve signal and the virtual convulsive cranial nerve signal as learning data.

Thereafter, in operation S14, the learning unit may generate and learn a user-customized convulsion detection and prediction algorithm by using the generated virtual normal cranial nerve signal and the virtual cranial nerve signal. In operation S15, the learning unit may determine how accurate the algorithm is through cranial nerve signals periodically provided from the epilepsy measuring device 100, and may achieve a high level of detection and prediction accuracy by implementing a convulsion detection and prediction algorithm optimized for each individual through the above series of processes.

In the above description, the case in which the learning unit generates a user-customized convulsion detection and prediction algorithm using cranial nerve signals has been mainly described. However, it goes without saying that the learning unit may generate an algorithm using multiple biological signals such as motion signals or electrocardiogram signals as well as cranial nerve signals.

In addition, the learning unit may generate a treatment stimulation signal generating algorithm by receiving cranial nerve signals before and after treatment stimulation applied through the stimulation unit 113 of the epilepsy measuring device 100, and learning based on this. In other words, the learning unit may extract features from cranial nerve signals before and after treatment stimulation, learn to generate a stimulation signal having the smallest separation width, and generate a treatment stimulation signal generating algorithm.

The learning unit learns classification criteria based on deep learning, and deep learning is defined as a set of machine learning algorithms that attempt high-level abstractions (summarizing key content or functions in large amounts of data or complex materials) through a combination of several nonlinear transformation methods. The learning unit may use any one of, for example, Deep Neural Networks (DNN), Convolutional Neural Networks (CNN), a Recurent Neural Network (RNN), and Deep Belief Networks (DBN) from among deep learning models.

The learning unit may use an algorithm and/or method (technique) such as Logistic regression, a Decision tree, a Nearest-neighbor classifier, Kernel discriminate analysis, a Neural network, a Support Vector Machine, a random forest, and a boosted tree to classify cranial nerve signals.

The learning unit may use an algorithm and/or method (technique) such as Linear regression, a Regression tree, Kernel regression, support vector regression, deep learning to predict convulsions or generate an appropriate treatment stimulus.

In addition, the learning unit may use an algorithm and/or method (technique) such as Principal component analysis, Non-negative matrix factorization, Independent component analysis, Manifold learning, and SVD for vector operation.

The learning unit may use an algorithm and/or method (technique) such as k-means, hierarchical clustering, mean-shift and self-organizing maps (SOMs) to group information.

The learning unit may use an algorithm and/or method (technique) such as Bipartite cross-matching, n-point correlation two-sample testing, and minimum spanning tree for data comparison.

However, the above-described algorithm and/or method (technique) are exemplary and the spirit of the present invention is not limited thereto.

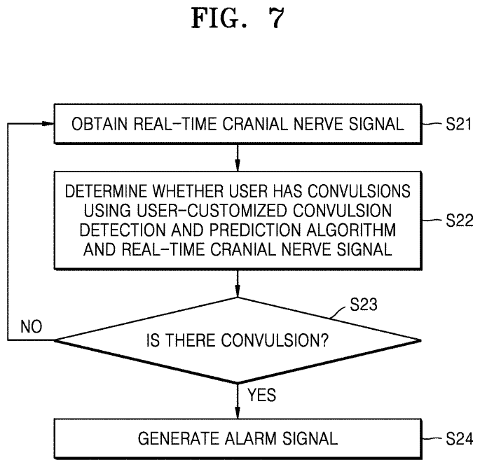

Hereinafter, with reference to FIG. 7, a process of determining whether convulsions occur in the determination unit 123 will be described.

FIG. 7 is a view for explaining a process of determining whether convulsions occur using the determination unit 123.

Referring to FIG. 7, first, in operation S21, the determination unit 123 obtains a cranial nerve signal measured in real time from the sensor unit 112 of the first unit 110. The determination unit 123 is arranged in the second unit 120 of the epilepsy measuring device 100, and a user-customized convulsion detection and prediction algorithm generated by the learning unit may be stored in advance.

Thereafter, in operations S22 and S23, the determination unit 123 may determine whether a user has convulsions according to the cranial nerve signal using the user-customized convulsion detection and prediction algorithm, and in operation S24, the determination unit 123 may generate an alarm signal when determining that convulsions occur.

Meanwhile, the determination unit 123 may generate a brain stimulation signal to apply a cranial nerve treatment stimulus corresponding to a brain stimulation signal to the user's brain and provide the brain stimulation signal to the stimulation unit 113. The determination unit 123 may store a cranial nerve treatment stimulation generating algorithm generated by the learning unit in advance, and may generate a brain stimulation signal so that the user's optimal treatment stimulation may be applied to the brain by using the cranial nerve treatment stimulation generating algorithm.

The above-described embodiments according to the present invention may be implemented in the form of a computer program that can be executed by various components on a computer, and such a computer program may be recorded on a computer-readable medium. Such a computer program may be recorded in a computer-readable medium. In this case, the medium may be to store a program executable by a computer. Examples of the medium may include a magnetic medium such as a hard disk, floppy disk, and magnetic tape, a magneto-optical medium such as a floppy disk, and one configured to store program instructions, including ROM, RAM, flash memory, and the like.

Meanwhile, the computer program may be particularly designed and structured for the present invention or available to those skilled in computer software. Examples of the program commands may include advanced language code that can be executed by a computer by using an interpreter or the like as well as machine language code made by a compiler.

As described above, an epilepsy monitoring device and an epilepsy measurement system according to embodiments of the present invention have the advantage that a battery can be replaced without additional surgical treatment through a dual structure of a first unit and a second unit, and medical imaging such as magnetic resonance imaging is possible even when the epilepsy monitoring device is inserted into the user's brain.

In addition, an epilepsy monitoring device and an epilepsy monitoring system according to embodiments of the present invention may achieve a high level of detection and prediction accuracy by generating a user-customized convulsion detection and prediction algorithm through artificial intelligence, and periodically updating the algorithm through provided data.

As described above, the present invention has been described with reference to an embodiment shown in the drawings, but this is only exemplary, and those of ordinary skill in the art will understand that various modifications and variations of the embodiment are possible therefrom. Therefore, the true technical protection scope of the present invention should be determined by the technical spirit of the appended claims.

### INDUSTRIAL APPLICABILITY

According to an embodiment of the present invention, an epilepsy monitoring device and an epilepsy monitoring system are provided. In addition, embodiments of the present invention may be applied to industrially used brain disease measurement technology.

1. An epilepsy monitoring device comprising:
a first unit comprising:
a first body portion arranged on the head of a user,
a sensor unit connected to the first body portion and including a first sensor configured to measure a cranial nerve signal, and
a stimulation unit connected to the first body portion and configured to apply cranial nerve treatment stimulation to the brain of the user according to a provided brain stimulation signal; and
a second unit electrically connected to the first unit and comprising:
a second body portion arranged on a body other than the head of the user,
a battery unit arranged in the second body portion and configured to supply power to the first unit, and
a communication unit for wireless communication with an external device.

2. The epilepsy monitoring device of claim 1, wherein the second unit further comprises a determination unit configured to determine whether the user has convulsions according to the cranial nerve signal using a previously stored user-customized convulsion detection and prediction algorithm, and generate an alarm signal when it is determined that the user has convulsions.

3. The epilepsy monitoring device of claim 2, wherein the determination unit is configured to generate the brain stimulation signal to apply the cranial nerve treatment stimulation corresponding to the brain stimulation signal to the brain of the user, and provide the brain stimulation signal to the stimulation unit.

4. The epilepsy monitoring device of claim 3, wherein the communication unit comprises:
first communication means configured to communicate with an external device by a remote active communication method; and
second communication means configured to communicate with an external device by a proximity passive communication method.

5. The epilepsy monitoring device of claim 4, wherein:
the first communication means is configured to transmit and receive the alarm signal to an external device by a remote active communication method, and
the second communication means is configured to transmit and receive the cranial nerve signal or the brain stimulation signal to an external device by a proximity passive communication method.

6. The epilepsy monitoring device of claim 1, wherein the sensor unit further comprises a second sensor configured to detect a biological signal different from the cranial nerve signal.

7. The epilepsy monitoring device of claim 6, wherein the second sensor is configured to detect a motion signal of the user.

8. An epilepsy monitoring system comprising:
a first unit comprising:
a first body portion arranged on the head of a user,
a sensor unit connected to the first body portion and including a first sensor configured to measure a cranial nerve signal, and
a stimulation unit connected to the first body portion and configured to apply cranial nerve treatment stimulation to the brain of the user according to a provided brain stimulation signal;
a second unit electrically connected to the first unit and comprising:
a second body portion arranged on a body other than the head of the user,
a battery unit arranged in the second body portion and configured to supply power to the first unit, and
a communication unit for wireless communication with an external device;
a learning unit configured to machine learn a user-customized convulsion detection and prediction algorithm using a normal cranial nerve signal and a convulsive cranial nerve signal from among pre-obtained cranial nerve signals of the user; and
a determination unit configured to determine whether the user has convulsions using the user-customized convulsion detection and prediction algorithm and the cranial nerve signals measured in real time by the sensor unit.

9. The epilepsy monitoring system of claim 8, wherein the determination unit is arranged in the second unit, and configured to generate an alarm signal when it is determined that the user has convulsions.

10. The epilepsy monitoring system of claim 9, wherein the determination unit is configured to generate the brain stimulation signal to apply the cranial nerve treatment stimulation corresponding to the brain stimulation signal to the brain of the user, and provide the brain stimulation signal to the stimulation unit.

11. The epilepsy monitoring system of claim 10, wherein the communication unit comprises:
first communication means configured to communicate with an external device by a remote active communication method; and
second communication means configured to communicate with an external device by a proximity passive communication method.

12. The epilepsy monitoring system of claim 11, wherein:
the first communication means is configured to transmit and receive the alarm signal to an external device by a remote active communication method, and
the second communication means is configured to transmit and receive the cranial nerve signal or the brain stimulation signal to an external device by a proximity passive communication method.

13. The epilepsy monitoring system of claim 8, wherein the sensor unit further comprises a second sensor configured to detect a biological signal different from the cranial nerve signal.

14. The epilepsy monitoring system of claim 13, wherein the second sensor is configured to detect a motion signal of the user.
